(19) United States
(12) Patent Application Publication
(10) Pub. No.: US 2010/0023124 A1
(43) Pub. Date: Jan. 28, 2010

(54) Biological nasal bridge implant and method of manufacturing

(76) Inventors: Guo-Feng Xu, Guangzhou (CN); Bin Xu, Guangzhou (CN)

Correspondence Address: Raymond Sun, 12420 Woodhall Way, Tustin, CA, 92782, US

(21) Appl. No.: 12/284,816

(22) Filed: Sep. 25, 2008

(30) Foreign Application Priority Data
Jul. 22, 2008 (CN) 200810029656.0

(51) Int. Cl. A61F 2/18; A61B 19/00; A61F 2/08; A61K 35/32

(52) U.S. Cl. 623/10; 128/898; 424/423; 424/548

(57) ABSTRACT

A nasal bridge implant is made according to a method that includes the steps of collecting animal material from a bovine or porcine source, the animal material being either a tendon or a ligament, removing cells from the animal material, shaping the animal material to provide a desired shape for the nasal bridge implant, crosslinking the animal material, removing antigens from the animal material, subjecting the animal material to an alkaline treatment, coupling into the animal material active substances which are capable of adhering growth factor and stem cell, and packing the animal material in a container that contains a sterilization solution.

### BACKGROUND OF THE INVENTION

1. Field of the Invention

The present invention relates to a medical prosthesis for human implantation, and in particular, to a biological nasal bridge implant used for nasal bridge augmentation surgery.

2. Description of the Prior Art

Nasal augmentation surgery is the most commonly seen surgery in the field of plastic surgery, and the implants that are currently being used to augment the bridge of the nose are all composed of silicone or Teflon. Although these two materials are biologically inert and may peacefully coexist with the human body after implantation, their composition and structure are not at all similar to the human body so they cannot become part of the host tissue. As a result, it is easy for the implants to shift position, abrade and corrode the skin, and for the implant to be detectable through careful observation, among other defects.

Thus, there still remains a need for a nasal bridge implant which avoids the drawbacks described above.

### SUMMARY OF THE DISCLOSURE

In order to accomplish the objects of the present invention, the present invention provides a nasal bridge implant made according to a method that comprises the following steps:

collecting animal material from a bovine or porcine source, the animal material being either a tendon or a ligament;

removing cells from the animal material;

shaping the animal material to provide a desired shape for the nasal bridge implant;

crosslinking the animal material;

removing antigens from the animal material;

subjecting the animal material to an alkaline treatment;

coupling into the animal material active substances which are capable of adhering growth factor and stem cell; and

packing the animal material in a container that contains a sterilization solution.

### BRIEF DESCRIPTION OF THE DRAWINGS

FIG. 1 is a plan view of a nasal bridge implant according to one embodiment of the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention relates to a biological model nasal bridge implant that is processed and formed using animal tendons/ligaments as the raw material. The raw material is first purified and processed, the cells are removed, and then the material is fixed using epoxy. Thereafter, multifold antigen removal technology, tissue induction technology and a series of other biochemical technological processes are applied. In composition and construction, the nasal bridge implant of the present invention is similar to human tissue, has good biocompatibility and high stability, is not easily degraded, is passively degraded only when the host tissue starts growing in, and does not initiate an immunological rejection response. The implant of the present invention is also able to induce tissue regeneration, to grow together with the host tissue, and to gradually convert itself into host tissue. The implant feels real, and will not cause position shifting, skin abrasion and corrosion.

The present invention provides a preparation method for a biological nasal bridge implant, the nasal bridge implant being formed using animal (preferably bovine or porcine) tendon/ligament as the raw material. The steps of preprocessing, cell removal, shaping, crosslinking and fixation, multifold antigen removal, Alkaline treatment, surface modification with active layer to induce activity, and radiation sterilization, are then applied to the raw material. The specific technical workflow process for preparation is as follows:
1. Preprocessing of animal tendon/ligament
2. Cell removal
3. Shaping/formation
4. Crosslinking
5. Antigen removal
6. Alkaline treatment
7. Surface modification with active layer to induce activity
8. Packaging and Sterilization

Step 1: In step 1 above, animal tendons/ligaments whose basic ingredient is collagen fiber are collected. Preferably, the tendons/ligaments are collected from bovine or porcine sources using techniques that are well-known in the art. Wide-spectrum disinfectants are used to saturate and disinfect the raw material, excess tissue and foreign substances are removed, and then the material is trimmed into a desired size/length that can be further shaped at step 3 below.

Step 2: In the cell removal step, an enzymatic or detergent (surfactant) elution method is used to remove all types of cells from the raw material (tendon or ligament). The enzymes, which can be trypsin and/or pepsin, are used for the enzymolysis of cell. The surfactants, which can be Triton X100, Tween-20, or emulsifier OP-10, are used to breakdown and wash off cell walls.

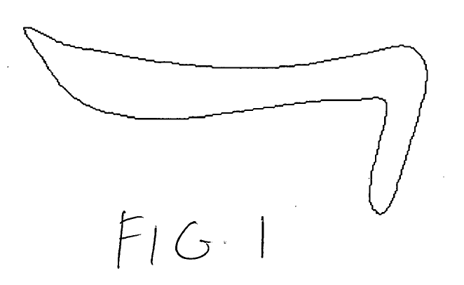

Step 3: In the shaping/formation step, the desired shape for the nasal bridge prosthetic, such as that shown in FIG. 1, is formed by further processing that is well-known in the art.

Step 4: The crosslinking and fixation step includes using a crosslinking fixative that uses collagen proteins for crosslinking, which makes the raw material stable. The crosslinking fixative that is used can be the epoxy compound having the following formula:

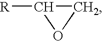

where R=CₙH₂ₙ₊₁ group or

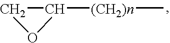

n=0, 1, 2, 3 . . . 12. The reagent concentration is 0.1-1N. The reaction temperature is selected between 0-45° C. (preferably no higher than 50° C.), and the reaction time may be selected between 2 and 96 hours.

Step 5: According to modern immunological theory, the antigenicity of animal tissues stems mainly from active groups located at specific sites and in specific conformations, and these active groups include —H₂*, —OH*, —SH*, etc. The specific conformations result mainly from some specific hydrogen bonding formed by spiral protein chains. The specific sites and conformations are called antigen determinants. The antigen removal step uses multiple reagents to block the active groups and alter the special conformation. The reagents used to block specific active groups are mainly nucleophilic reagents that react easily with —H₂*, —OH*, —SH* and other similar groups. These reagents include carboxylic acid anhydrides, acyl chlorides, acylamides, epoxy compounds, etc. The reagents that can be used to alter specific conformations include class one strong hydrogen bond formation agents, such as guanidine hydrochloride. Because the specific conformations result mainly from some specific hydrogen bonding formed by spiral protein chains, using strong hydrogen bond formation agents to replace the specific hydrogen bond makes it possible to change the specific conformation. Here the * symbol on the groups indicates that they are a small number of specific groups which are located in specific locations and are able to produce a response to immune signals, and they are not the standard —NH₂, —OH, —SH groups. These specific groups are in a high-energy activity state, preferable for nucleophilic reagent initiated reactions, just as the catalyst's active center is preferable for the reactant or toxin reaction.

Step 6: the alkaline treatment is mainly designed for destroying possibly existing prions. For example, 1-4N sodium hydroxide solution can be used to immerse the prosthesis for 60 minutes at a temperature of 35±2° C. Such processing has already been proven in numerous studies to be effective in destroying prions.

Step 7: In step 7 above, the surface modification includes a process of coupling active substances which are capable of adhering growth factor and stem cell into the prosthesis material, so the prosthesis can adhere and enrich growth factor and stem cell released from human body's self-repair mechanism after implantation, thereby promoting growth factor and stem cell for highly effective expression in the prosthesis over a long period of time, and inducing the stem cells to differentiate into repair tissue mother cells, to again divide and proliferate, regenerate new tissue, and ultimately become autologous nasal bridge tissue. The active substances introduced can be a specific polypeptide or glycosaminoglycan compound. Here the specific polypeptides are mainly formed of 16 lysine oligopeptides with arginine, glycine, aspartic acid and other components, for example, a polypeptide constructed of lysine (16)—glycine-arginine-glycine-aspartic acid-serine-proline-cysteine, with the glycosaminoglycan compound being mainly hyaluronic acid, chondroitin sulfate, cortisone sulfate, keratin sulfate, heparin, heparin sulfate, etc. The method of introduction may be accomplished by coupling, chemical adsorption, physical adsorption, or collagen film encapsulation. Coupling is preferred, and coupling agents that may be used are internal carboxylic diacid anhydrides, diacyl dichlorides, diacyl diamides, carbodiimides, and diepoxides.

Step 8: In the packaging and sterilization step, the prosthesis is sealed in a dual-layer plastic bag containing physiological saline storage solution. The packed product is then sterilized under minimum 25 kGy γ-irradiation. This sterilization method has been proven to kill known pathogens, except prions.

Compared to the conventionally-available silicone and Teflon nasal bridge implants, the advantages of the present invention for a biological model nasal bridge implant lie in the fact that it is produced from a purely natural material, its composition is basically similar to that of human tissue, it possesses good biocompatibility, and has no immunological rejection response. After implantation, the implant of the present invention can induce the host tissue to grow into the implant and to heal with the host tissue into one piece, it feels real, there are no irritating foreign materials, and it cannot shift position, corrode or be exposed to the outside, or suffer other complications.

### EXAMPLE

Obtain fresh and healthy animal tendon/ligament, place in 0.1% benzalkonium bromide sterilization fluid and saturate for 60 min, then remove foreign substances, repair and trim into the desired size and length. Thereafter, remove, clean, place in trypsin-Tris hydrochloride buffer at room temperature to perform enzymolysis for 2-24 hours. Thereafter, remove, rinse with water, place in a 1% OP-10 solution containing 1 μM benzyl fluorosulfide protease inhibitor, saturate for 8 hours, and remove again. While stirring, use water to rinse three times, remove again, leach out the water content, and create the desired structural shapes and forms such as shown in FIG. 1. Place in a fixation reactor and use a crosslinking agent to perform crosslinking fixation, with the crosslinking agent selected from the epoxy compound described above, or adipoyl chloride with concentration between 0.1-1N, and react at room temperature for 2-96 hours. After the crosslinking reaction is complete, remove, clean, place in the antigen reactor, add one of the above-described nucleophilic reagents, and react at room temperature for 10-16 hours. Select two different types of reagents and react twice. Then use guanidine hydrochloride solution to react once at a temperature between 5-30° C. with a reaction time of 8-24 hours. Remove, clean, place in 1-24 sodium hydroxide solution at 30-35° C., saturate and process for 60 minutes, and then discard the reaction fluid. Use diluted acid to neutralize the residual sodium hydroxide, and then clean. Place in the special-use reactor for surface modification, add the lysine (16)—glycine-arginine-glycine-aspartic acid-serine-proline-cysteine polypeptide and the adipoyl chloride coupling reagent, then react in moderate conditions for 8-16 hours at 25±2° C. Remove, and wash thoroughly. Seal using physiological saline storage solution in a dual-layer plastic bag, send for radiation sterilization, and obtain the finished product.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method of preparing a nasal bridge implant, comprising:
collecting animal material from a bovine or porcine source, the animal material being either a tendon or a ligament;
removing cells from the animal material;
shaping the animal material to provide a desired shape for the nasal bridge implant;
crosslinking the animal material;
removing antigens from the animal material;
subjecting the animal material to an alkaline treatment;
coupling into the animal material active substances which are capable of adhering growth factor and stem cell; and
packing the animal material in a container that contains a sterilization solution.

2. The method of claim 1, wherein the cell removal step uses an enzymatic method or a detergent elution method to remove cells.

3. The method of claim 2, wherein the enzymatic method uses trypsin or pepsin to perform enzymatic action.

4. The method of claim 2, wherein the detergents used in the detergent elution method are Triton X100, Tween-20, and emulsifier OP-10.

5. The method of claim 1, wherein the crosslinking step is implemented using the epoxy compound 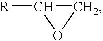
R=CₙH₂ₙ₊₁ group or 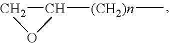
n=0, 1, 2, 3 . . . 12, as the crosslinking agent.

6. The method of claim 1, wherein the antigen removal step uses nucleophilic reagents and strong hydrogen bond formation agents that easily activate a hydrogen reaction with —NH₂, —OH, —SH and other groups to block specific groups and to change specific conformations.

7. The method of claim 6, wherein the nucleophilic reagents include carboxylic acid anhydrides, acyl chlorides, acylamides, and epoxides.

8. The method of claim 6, wherein the strong hydrogen bonding agents includes guanidine compounds.

9. The method of claim 1, wherein the alkaline treatment step uses 1-4N sodium hydroxide to immerse the animal material for a fixed period of time.

10. The method of claim 1, wherein the active substances are polypeptides containing 16 lysine oligopeptides with arginine, glycine, and aspartic acid.

11. A nasal bridge implant made according to a method that comprises the following steps:
collecting animal material from a bovine or porcine source, the animal material being either a tendon or a ligament;
removing cells from the animal material;
shaping the animal material to provide a desired shape for the nasal bridge implant;
crosslinking the animal material;
removing antigens from the animal material;
subjecting the animal material to an alkaline treatment;
coupling into the animal material active substances which are capable of adhering growth factor and stem cell; and
packing the animal material in a container that contains a sterilization solution.

12. The implant of claim 11, wherein the cell removal step uses an enzymatic method or a detergent elution method to remove cells.

13. The implant of claim 11, wherein the crosslinking step is implemented using the epoxy compound 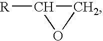
R=CₙH₂ₙ₊₁ group or 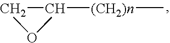
n=0, 1, 2, 3 . . . 12, as the crosslinking agent.

14. The implant of claim 11, wherein the antigen removal step uses nucleophilic reagents and strong hydrogen bond formation agents that easily activate a hydrogen reaction with —NH₂, —OH, —SH and other groups to block specific groups and to change specific conformations.

15. The implant of claim 11, wherein the alkaline treatment step uses 1-4N sodium hydroxide to immerse the animal material for a fixed period of time.

16. The implant of claim 11, wherein the active substances are polypeptides containing 16 lysine oligopeptides with arginine, glycine, and aspartic acid.
